(19) United States
(12) Patent Application Publication
(10) Pub. No.: US 2025/0169711 A1
(43) Pub. Date: May 29, 2025

(54) COIL UNIT

(71) Applicant: FUJIFILM Corporation, Tokyo (JP)

(72) Inventors: Koichi ARAI, Tokyo (JP); Hisaaki OCHI, Tokyo (JP); Yosuke OTAKE, Tokyo (JP); Kojiro IWASAWA, Tokyo (JP); Masayoshi DOHATA, Tokyo (JP)

(73) Assignee: FUJIFILM Corporation, Tokyo (JP)

(21) Appl. No.: 18/963,192

(22) Filed: Nov. 27, 2024

(30) Foreign Application Priority Data
Nov. 28, 2023 (JP) 2023-200505

(51) Int. Cl. A61B 5/055

(52) U.S. Cl. CPC A61B 5/055

(57) ABSTRACT

Provide is a coil unit capable of easily fixing a receive coil to a patient. A coil unit according to the present disclosure includes a receive coil that receives a magnetic resonance signal of an examination target, a belt member with one end side fixed to the receive coil and the other end side being a free end, and a locking member that is fixed to the receive coil and fixes the belt member at a position of any length from the one end side of the belt member, in which a user winds the belt member fixed to the receive coil around a trunk of a patient and fixes the belt member by the locking member such that the receive coil is fixed to the patient.

### CROSS-REFERENCE TO RELATED APPLICATION

The present application claims priority under 35 U.S.C § 119(a) to Japanese Patent Application No. 2023-200505 filed on Nov. 28, 2023, which is hereby expressly incorporated by reference, in its entirety, into the present application.

### BACKGROUND OF THE INVENTION

### 1. Field of the Invention

The present invention relates to a coil unit, and particularly to a technique for fixing a receive coil for receiving a magnetic resonance signal to a trunk of a patient.

### 2. Description of the Related Art

In a case where abdominal imaging is performed in a magnetic resonance imaging (MRI) examination, it is necessary to fix an abdominal coil to an abdomen of a patient so that the abdominal coil does not shift. JP2012-152465A, JP1992-2346A (JP-H4-2346A), and JP1990-6007U (JP-H2-6007U) disclose a technique of fixing a patient to an examination table by intersecting belts and fixing the belts.

### SUMMARY OF THE INVENTION

However, in the related art, in a case in which a patient is fixed separately from the installation of the abdominal coil, the patient, the abdominal coil, and the belt are fixed separately from each other. Therefore, it remains a problem to fix the abdominal coil with the patient.

The present invention has been made in a view of such circumstances, and an object of the present invention is to provide a coil unit with which a receive coil can be easily fixed to a patient.

In order to achieve the above object, according to a first aspect of the present disclosure, a coil unit comprises a receive coil that receives a magnetic resonance signal of an examination target, a belt member with one end side fixed to the receive coil and the other end side being a free end, and a locking member that is fixed to the receive coil and fixes the belt member at a position of any length from the one end side of the belt member.

According to the present aspect, the receive coil can be easily fixed to the patient by winding the belt member fixed to the receive coil around the trunk of the patient and fixing the belt member with the locking member.

In a second aspect of the present disclosure, the coil unit according to the first aspect further preferably comprises a loop member that is fixed to the receive coil and restricts a path of the belt member.

In a third aspect of the present disclosure, the coil unit according to the second aspect, in which the one end side of the belt member, the locking member, and the loop member are preferably disposed in a straight line.

In a fourth aspect of the present disclosure, the coil unit according to the first to third aspects, in which the belt member preferably includes a first belt member of which one end side is fixed to the receive coil and the other end side is a free end, and a second belt member of which one end side is fixed to the receive coil and the other end side is a free end, and the locking member preferably includes a first locking member that fixes the first belt member and a second locking member that fixes the second belt member.

In a fifth aspect of the present disclosure, the coil unit according to the fourth aspect further preferably comprises a first loop member that is fixed to the receive coil and restricts a path of the first belt member, and a second loop member that is fixed to the receive coil and restricts a path of the second belt member.

In a sixth aspect of the present disclosure, the coil unit according to the fifth aspect, in which a straight line connecting the one end side of the first belt member and the first loop member preferably intersects with a straight line connecting the one end side of the second belt member and the second loop member.

In a seventh aspect of the present disclosure, the coil unit according to any one of the first to sixth aspects, in which the receive coil preferably has a structure that encircles around a trunk of a patient.

In an eighth aspect of the present disclosure, the coil unit according to any one of the first to seventh aspects, further preferably comprises a pipe member into which the belt member is inserted.

In a ninth aspect of the present disclosure, the coil unit according to any one of the first to eighth aspects, in which the locking member is preferably fixed to a center of the receive coil.

In a tenth aspect of the present disclosure, the coil unit according to any one of the first to ninth aspects, in which the belt member is preferably a cable that outputs the magnetic resonance signal received by the receive coil.

In an eleventh aspect of the present disclosure, the coil unit according to the tenth aspect, in which the belt member is preferably a flat cable in which a plurality of cables are bundled in a planar shape.

According to the present invention, it is possible to easily fix a receive coil to a patient.

### BRIEF DESCRIPTION OF THE DRAWINGS

FIG. 1 is a perspective view showing an example of an MRI apparatus to which a coil unit is applied.

FIG. 2 is a perspective view of an abdominal coil unit according to a first embodiment.

FIG. 3 is a top view of a patient placed on a top plate.

FIG. 4 is a schematic view viewed from an arrow A of FIG. 3.

FIG. 5 is a perspective view of an abdominal coil unit according to a second embodiment.

FIG. 6 is a top view of a patient placed on a top plate.

FIG. 7 is a schematic view viewed from an arrow B of FIG. 6.

FIG. 8 is a perspective view of an abdominal coil unit according to a third embodiment.

FIG. 9 is a top view of a patient placed on a top plate.

FIG. 10 is a schematic view viewed from an arrow C of FIG. 9.

FIG. 11 is a top view of a patient placed on a top plate.

FIG. 12 is a schematic view viewed from an arrow D of FIG. 11.

FIGS. 13A to 13C are diagrams showing coil units according to a fifth embodiment.

FIG. 14 is a partially enlarged view of a pipe.

### DESCRIPTION OF THE PREFERRED EMBODIMENTS

Hereinafter, preferred embodiments of a coil unit according to the present disclosure will be described with reference to the accompanying drawings. In the present specification, the same reference numeral will be given to the same configuration element and the duplicate description thereof will be omitted as appropriate.

### MRI Apparatus

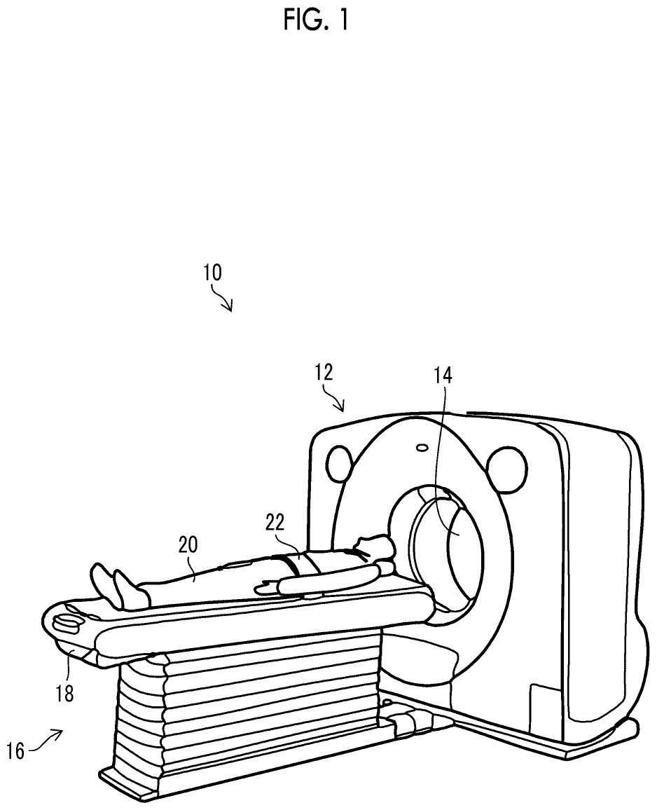

FIG. 1 is a perspective view showing an example of an MRI apparatus 10 to which a coil unit according to the present disclosure is applied. As shown in FIG. 1, the MRI apparatus 10 comprises a gantry 12 and an examination table 16. The gantry 12 has a bore 14 which is an examination (imaging) space on a cylinder. In addition, the gantry 12 has an MRI magnet and various other coils disposed therein.

The examination table 16 is provided to face the bore 14 on a front side of the gantry 12. The examination table 16 comprises a top plate 18. By moving the top plate 18 in the bore 14, an examination site (an example of an “examination target”) of a patient 20 to be imaged placed on the top plate 18 is set as a center of a static magnetic field in the bore 14.

A multi-channel radio frequency (RF) receive coil 22 (hereinafter, referred to as a coil 22) consisting of a plurality of element coils for receiving a magnetic resonance signal (free induction decay (FID)) generated in the patient 20 is mounted on the examination site of the patient 20. In the example shown in FIG. 1, the abdomen of the patient 20 is an examination site, and the coil 22 is mounted on the abdomen of the patient 20.

A reception side cable (not shown) that outputs the magnetic resonance signal received by the coil 22 is connected to the coil 22. A reception side connector (not shown) is connected to an end part of the reception side cable. The reception side connector is connected to an examination table-side connector of an examination table-side cable (not shown). Accordingly, the reception side cable and the examination table-side cable are communicably connected to each other through the connectors.

The examination table-side cable is stored in a cable storage portion (not shown) of the examination table 16. The magnetic resonance signal of the examination target received by the coil 22 is transmitted to a signal processing unit (not shown) through the reception side cable and the examination table-side cable. The signal processing unit performs signal processing on the received magnetic resonance signal to convert the magnetic resonance signal into an image signal.

### First Embodiment

### Configuration of Coil Unit

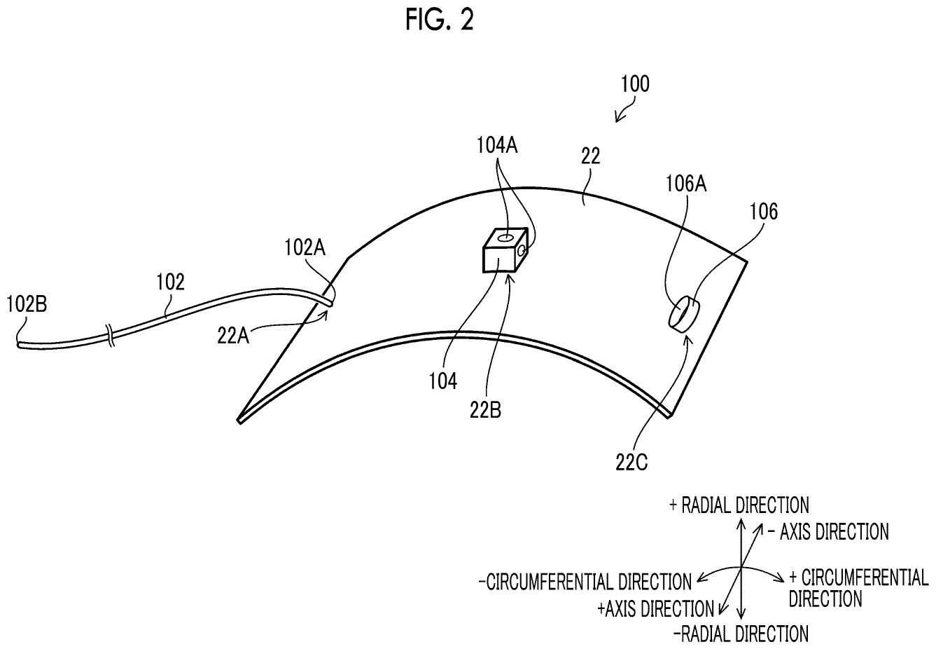

FIG. 2 is a perspective view of an abdominal coil unit 100 according to the first embodiment. The abdominal coil unit 100 has a mechanism for fixing the coil 22 to the abdomen of the patient 20. In FIG. 2, a trunk direction of the patient 20 (not shown in FIG. 2) to which the coil 22 is fixed is defined as a ±axis direction, a direction along a circumference of the trunk of the patient 20 is defined as a ±circumferential direction, and a direction orthogonal to the ±axis direction and the ±circumferential direction is defined as a ±radial direction.

As shown in FIG. 2, the abdominal coil unit 100 comprises a coil 22, a belt 102, a stopper 104, and a loop 106. The belt 102, the stopper 104, and the loop 106 are provided on a surface of the coil 22 on a +radial direction side (hereinafter, referred to as an upper surface of the coil 22).

The belt 102 (an example of a “belt member”) is a flexible elongated member, for example, a string-like member. The belt 102 may be a band-shaped member. One end part 102A (an example of “one end side”) of the belt 102 is fixed to a fixed point 22A of the coil 22, and the other end part 102B (an example of “the other end side”) is a free end. The position of the fixed point 22A is at the center of the upper surface of the coil 22 in the ±axis direction and on the −circumferential direction side in the ±circumferential direction. The position of the fixed point 22A may be an end part of the upper surface of the coil 22 on the −circumferential direction side. The position of the belt 102 fixed to the fixed point 22A is not limited to the end part 102A and may be any position on the belt 102 on the end part 102A side.

The stopper 104 (an example of a “locking member”) has a mechanism that allows the belt 102 to be attachably and detachably fixed at a position of any length from the end part 102A. The optional length position is not limited to a position of any continuous length, and may be a position of any of a plurality of discrete length positions.

The stopper 104 is fixed to a fixed point 22B of the coil 22. The position of the fixed point 22B is a center of the upper surface of the coil 22 in the ±axis direction and is a center of the upper surface in the ±circumferential direction. That is, the position of the fixed point 22B is the center of the upper surface of the coil 22. The position of the fixed point 22B may be on the +circumferential direction side or the −circumferential direction side of the upper surface of the coil 22.

The stopper 104 includes an insertion hole 104A that communicates from the +circumferential direction side to the +radial direction side and a locking mechanism (not shown). The stopper 104 movably inserts the belt 102 into the insertion hole 104A in a state where the locking mechanism is released. The stopper 104 fixes the belt 102 inserted into the insertion hole 104A in a state where the locking mechanism is locked. That is, the stopper 104 can fix the belt 102 at any position of the belt 102.

The loop 106 (an example of a “loop member”) is a member that restricts a path of the belt 102. Here, the loop 106 restricts the movement of the belt 102 in the ±axis direction and the ±radial direction. The loop 106 comprises an insertion hole 106A for inserting the belt 102 in a ±circumferential direction.

The loop 106 is fixed to a fixed point 22C of the coil 22. The position of the fixed point 22C is a center of the upper surface of the coil 22 in the ±axis direction and is on the +circumferential direction side of the coil 22 in the ±circumferential direction. The position of the fixed point 22C may be an end part of the upper surface of the coil 22 on the +circumferential direction side.

In this manner, on the upper surface of the coil 22, the end part 102A of the belt 102, the stopper 104, and the loop 106 are disposed in a straight line. In the present specification, the term “disposed in a straight line” is not limited to being disposed on a single exact straight line. That is, a case where the end part 102A of the belt 102, the stopper 104, and the loop 106 are disposed to be shifted from a straight line to the extent that the intended effect in the present disclosure can be obtained is also interpreted as “disposed in a straight line”.

### Mounting of Coil Unit

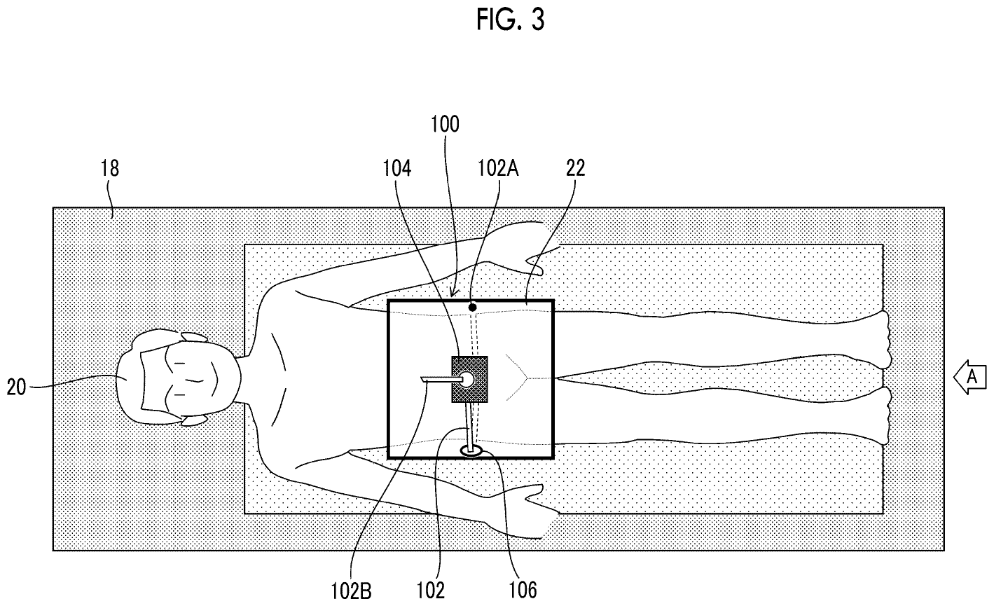

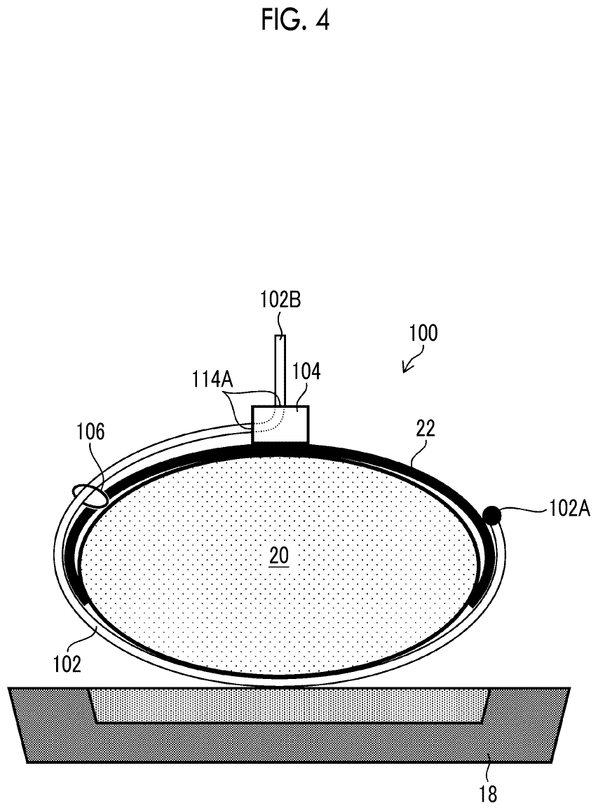

FIG. 3 is a top view of the patient 20 placed on the top plate 18. An abdominal coil unit 100 is mounted on an abdomen of a patient 20 shown in FIG. 3. In addition, in FIG. 3, the belt 102 on the back side of the patient 20 is shown by a dotted line. In addition, FIG. 4 is a schematic view viewed from an arrow A of FIG. 3.

In order to mount the abdominal coil unit 100 on the patient 20 as shown in FIGS. 3 and 4, first, the user winds the belt 102 through the lower surface side, which is the surface opposite to the upper surface of the coil 22, and inserts the belt 102 from the end part 102B into the insertion hole 106A of the loop 106. In addition, the user inserts the belt 102 from the end part 102B into the insertion hole 104A of the stopper 104 in the unlocked state. Subsequently, the user passes the body of the patient 20 through a space surrounded by the lower surface of the coil 22 and the belt 102, and brings the lower surface of the coil 22 into contact with the abdomen of the patient 20. The patient 20 may pass his or her body through the space surrounded by the lower surface of the coil 22 and the belt 102 from the head or the foot.

Then, the user pulls the end part 102B side of the belt 102 in the +radial direction of the coil 22 to bring the coil 22 into a state of sufficiently attaching to the abdomen of the patient 20, and brings the locking mechanism of the insertion hole 104A of the stopper 104 into the locked state. Accordingly, the belt 102 is fixed by the stopper 104, and the coil 22 is tightly fixed to the abdomen of the patient 20 by the belt 102.

The mounting of the abdominal coil unit 100 may be performed as follows. That is, the user aligns the coil 22 in a direction in which the ±axis direction of FIG. 2 is the trunk direction of the patient 20. Next, the user brings the lower surface of the coil 22 into contact with the abdomen of the patient 20.

Subsequently, the user winds the belt 102 from the back side of the patient 20 to the front side and inserts the belt 102 from the end part 102B into the insertion hole 106A of the loop 106. Further, the user inserts the belt 102 from the end part 102B into the insertion hole 104A of the stopper 104 in the unlocked state.

Then, the user pulls the end part 102B side of the belt 102 in the +radial direction of the coil 22 to bring the coil 22 into a state of sufficiently attaching against the abdomen of the patient 20, and brings the locking mechanism of the insertion hole 104A of the stopper 104 into the locked state.

Even with such a procedure, it is possible to mount the abdominal coil unit 100 on the patient 20.

In this manner, with the abdominal coil unit 100 according to the first embodiment, the belt 102 is fixed to at least one point of the coil 22, and the belt 102 is put on the patient 20. Therefore, the user can complete the fixing of the coil 22 only by pulling the belt 102.

Here, the user inserts the belt 102 into the insertion hole 106A of the loop 106, so that the movement of the belt 102 in the ±axis direction and the ±radial direction is restricted, and the belt 102 can be wound around the appropriate position. In addition, since the stopper 104 is disposed at the center of the coil 22, the user can complete the fixing of the coil 22 only by pulling the belt 102 disposed at a position where the upper surface of the coil 22 is easy to be gripped and held. Further, since the end part 102A of the belt 102, the stopper 104, and the loop 106 are disposed in a straight line, the user can efficiently transmit the pulling force of the belt 102 to the end part 102A of the belt 102, and the coil 22 can be appropriately fixed to the patient 20.

FIG. 3 shows a state in which the abdominal coil unit 100 is mounted in a direction in which the +axis direction side of the coil 22 is the head side of the patient 20, but the abdominal coil unit 100 may be mounted in a direction in which the −axis direction side of the coil 22 is the head side of the patient 20. In addition, here, although the abdominal coil has been described as an example of the trunk part coil, a target portion of the trunk part coil may be any part such as a head, a neck, a shoulder, an arm, a chest, a waist, or a leg.

### Second Embodiment

### Configuration of Coil Unit

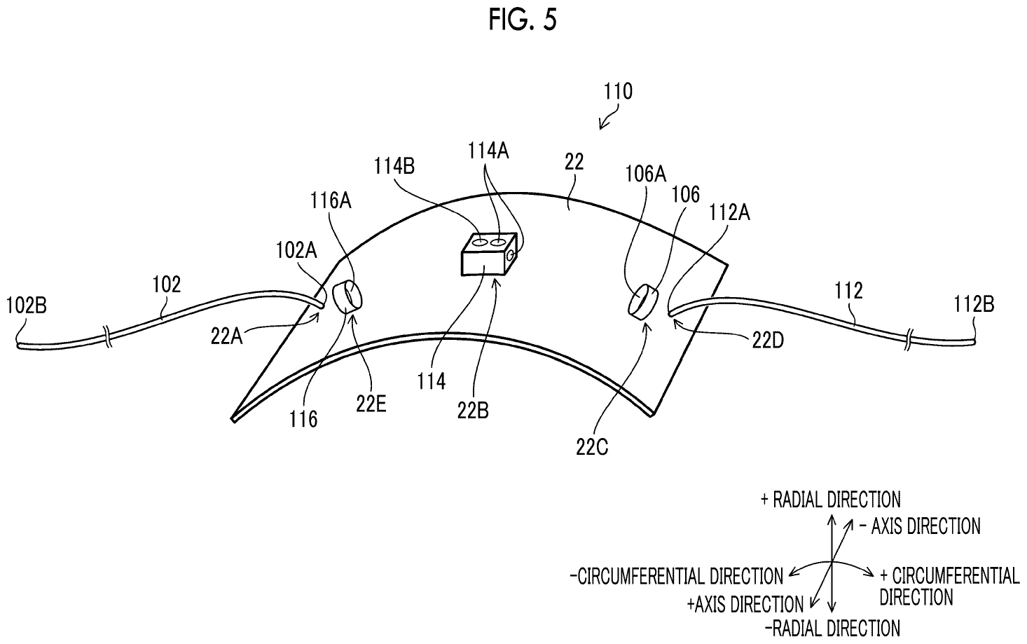

FIG. 5 is a perspective view of an abdominal coil unit 110 according to the second embodiment. The abdominal coil unit 110 comprises a coil 22, a belt 102, a loop 106, a belt 112, a stopper 114, and a loop 116.

The belt 112 (an example of a “second belt member”) is the same string-like member as the belt 102 (an example of a “first belt member”). One end part 112A of the belt 112 is fixed to a fixed point 22D of the coil 22, and the other end part 112B is a free end. The position of the fixed point 22D is a center of the upper surface of the coil 22 in the ±axis direction and is on the ±circumferential direction side of the upper surface in the +circumferential direction. The position of the fixed point 22D may be an end part of the upper surface of the coil 22 on the +circumferential direction side.

The stopper 114 (an example of the “first locking member” and an example of the “second locking member”) has a mechanism that allows the belt 102 to be attachably and detachably fixed at a position of any length from the end part 102A and a mechanism that allows the belt 112 to be attachably and detachably fixed at a position of any length from the end part 112A. The stopper 114 is fixed to the fixed point 22B of the coil 22. The position of the fixed point 22B is the center of the upper surface of the coil 22.

The stopper 114 includes an insertion hole 114A that communicates from the +circumferential direction side to the +radial direction side, an insertion hole 114B that communicates from the −circumferential direction side to the +radial direction side, and a locking mechanism (not shown) corresponding to each of the insertion hole 114A and the insertion hole 114B. The stopper 114 movably inserts the belt 102 into the insertion hole 114A in a state where the locking mechanism of the insertion hole 114A is released. The stopper 114 fixes the belt 102 inserted into the insertion hole 114A in a state where the locking mechanism of the insertion hole 114A is locked. Similarly, the stopper 114 movably inserts the belt 112 into the insertion hole 114B in a state where the locking mechanism of the insertion hole 114B is released. The stopper 114 fixes the belt 112 inserted into the insertion hole 114B in a state where the locking mechanism of the insertion hole 114B is locked. The stopper 114 may fix both the belt 102 inserted into the insertion hole 114A and the belt 112 inserted into the insertion hole 114B by a single locking mechanism.

The loop 116 (an example of a “second loop member”) is a member that restricts the movement of the belt 112 in the ±axis direction and the ±radial direction, similarly to the loop 106 (an example of a “first loop member”). The loop 116 comprises an insertion hole 116A for inserting the belt 112. The loop 116 is fixed to a fixed point 22E of the coil 22. The position of the fixed point 22E is a center of the upper surface of the coil 22 in the ±axis direction and is a −circumferential direction side of the coil 22 in the ±circumferential direction.

In this manner, on the upper surface of the coil 22, the end part 102A of the belt 102, the stopper 104, and the loop 106 are disposed in a straight line, and the end part 112A of the belt 112, the stopper 114, and the loop 116 are disposed in a straight line.

### Mounting of Coil Unit

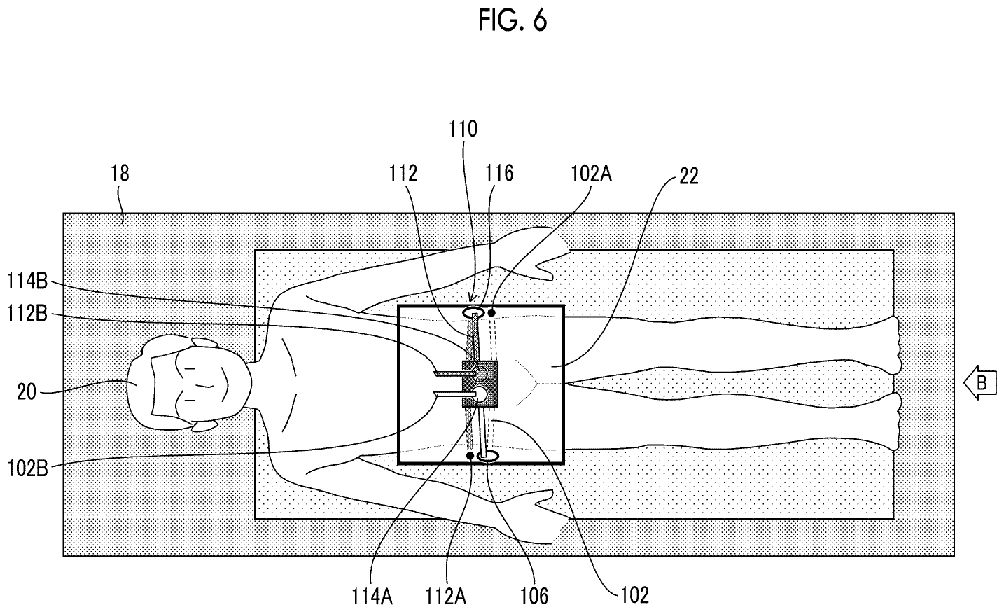

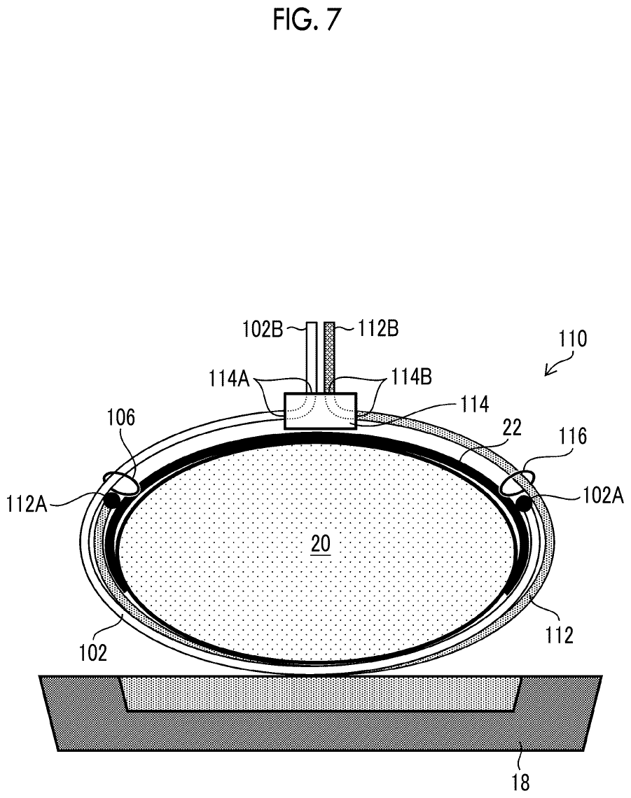

FIG. 6 is a top view of the patient 20 placed on the top plate 18. An abdominal coil unit 110 is mounted on an abdomen of a patient 20 shown in FIG. 6. In FIG. 6, the belt 102 and the belt 112 on the back side of the patient 20 are shown by a dotted line. In addition, FIG. 7 is a schematic view viewed from an arrow B of FIG. 6.

In order to mount the abdominal coil unit 110 on the patient 20 as shown in FIGS. 6 and 7, first, the user winds the belt 102 through the lower surface side of the coil 22 and inserts the belt 102 from the end part 102B into the insertion hole 106A of the loop 106. In addition, the user inserts the belt 102 from the end part 102B into the insertion hole 104A of the stopper 104 in the unlocked state.

Similarly, the user winds the belt 112 through the lower surface side of the coil 22 and inserts the belt 112 from the end part 112B into the insertion hole 116A of the loop 116. In addition, the user inserts the belt 112 from the end part 112B into the insertion hole 114B of the stopper 114 in the unlocked state. In this manner, the belt 102 and the belt 112 are wound around the lower surface side of the coil 22 in an opposite direction mutually.

Subsequently, the user passes the body of the patient 20 through a space surrounded by the lower surface of the coil 22 and the belt 102 and the belt 112, and brings the lower surface of the coil 22 into contact with the abdomen of the patient 20.

Then, the user pulls the belt 102 and the belt 112 in the +radial direction to bring the coil 22 into a state of sufficiently attaching to the abdomen of the patient 20, and brings the locking mechanism of the insertion hole 114A and the locking mechanism of the insertion hole 114B of the stopper 114 to the locked state. Accordingly, the belt 102 and the belt 112 are fixed by the stopper 114, and the coil 22 is tightly fixed to the abdomen of the patient 20 by the belt 102 and the belt 112.

Here, although the user fixes the belt 102 and the belt 112 by operating the stopper 114 once, the user may fix one of the belt 102 and the belt 112 and then fix the other. In addition, the user may mount the abdominal coil unit 110 on the patient 20 by aligning the coil 22 in the direction in which the ±axis direction of FIG. 5 is the trunk direction of the patient 20 and bringing the lower surface of the coil 22 into contact with the abdomen of the patient 20, and then winding the belt 102 and the belt 112 around the patient 20.

In this manner, with the abdominal coil unit 110 according to the second embodiment, the belt 102 and the belt 112 are fixed to at least two points of the coil 22, and the belt 102 and the belt 112 are put on the patient 20. Accordingly, fixing of the coil 22 to the trunk of the patient 20 can be completed equally to the right and left by simply pulling the belt 102 and the belt 112 located on the upper surface of the coil 22 at positions that are easily gripped and held.

Here, the belt 102 and the belt 112 are inserted into the insertion hole 106A of the loop 106 and the insertion hole 116A of the loop 116, respectively, so that the movement of the belt 102 and the belt 112 in the ±axis direction and the ±radial direction is restricted. Accordingly, the user can wind the belt 102 and the belt 112 at appropriate positions. In addition, since the end part 102A of the belt 102, the stopper 114, and the loop 106 are disposed in a straight line and the end part 112A of the belt 112, the stopper 114, and the loop 116 are disposed in a straight line, the user can efficiently transmit the pulling force of the belt 102 and the belt 112 to the end part 102A of the belt 102 and the end part 112A of the belt 112, and the coil 22 can be appropriately fixed to the patient 20. Further, the coil 22 can be fixed equally to the right and left sides of the trunk of the patient 20 by winding and fixing the belt 102 and the belt 112 from both sides of the patient 20 in an opposite direction mutually.

### Third Embodiment

### Configuration of Coil Unit

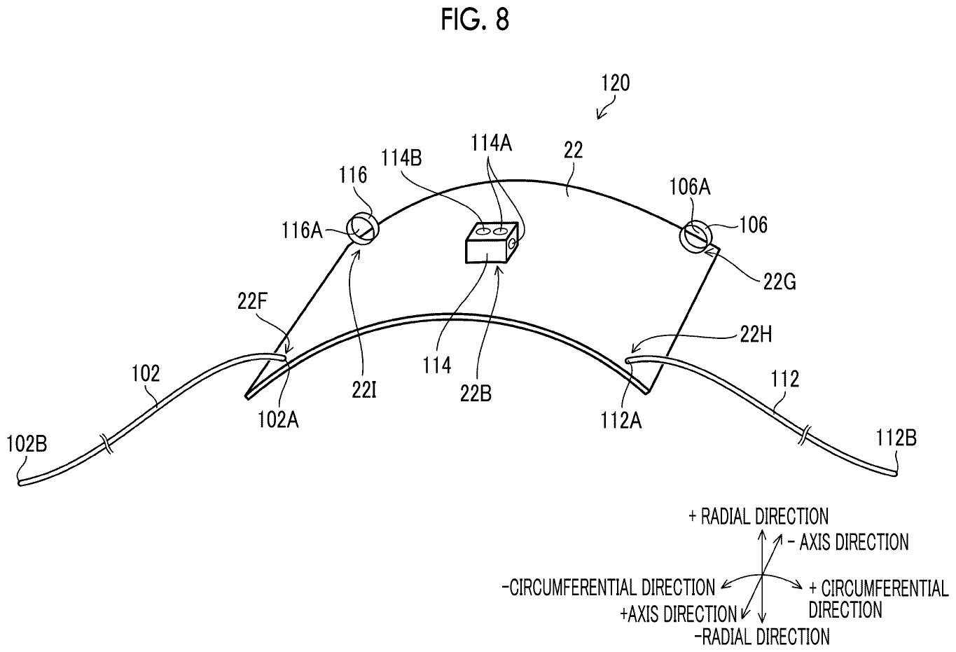

FIG. 8 is a perspective view of an abdominal coil unit 120 according to the third embodiment. The abdominal coil unit 120 comprises a belt 102, a loop 106, a belt 112, a stopper 114, and a loop 116, similarly to the abdominal coil unit 110.

The end part 102A of the belt 102 is fixed to a fixed point 22F of the coil 22. The position of the fixed point 22F is an end part of the upper surface of the coil 22 on the +axis direction side in the ±axis direction, and is on the −circumferential direction side in the ±circumferential direction of the upper surface.

The loop 106 is fixed to a fixed point 22G of the coil 22. The position of the fixed point 22G is an end part of the upper surface of the coil 22 on the −axis direction in the ±axis direction, and is on the ±circumferential direction side of the coil 22. In this manner, the end part 102A of the belt 102 and the loop 106 are disposed on a diagonal line of the upper surface of the coil 22.

The end part 112A of the belt 112 is fixed to a fixed point 22H of the coil 22. The position of the fixed point 22H is an end part of the upper surface of the coil 22 on the +axis direction side in the ±axis direction, and is on the +circumferential direction side in the ±circumferential direction of the upper surface.

The loop 116 is fixed to a fixed point 22I of the coil 22. The position of the fixed point 22I is an end part of the upper surface of the coil 22 on the −axis direction in the ±axis direction, and is on the −circumferential direction side in the ±circumferential direction of the coil 22. In this manner, the end part 112A of the belt 112 and the loop 116 are disposed on a diagonal line of the upper surface of the coil 22, and the diagonal line is different from the diagonal line of the end part 102A of the belt 102 and the loop 106.

The stopper 114 is fixed to the fixed point 22B that is a center of the upper surface of the coil 22.

In this manner, on the upper surface of the coil 22, the end part 102A of the belt 102, the stopper 114, and the loop 106 are disposed in a straight line. In addition, on the upper surface of the coil 22, the end part 112A of the belt 112, the stopper 114, and the loop 116 are disposed in a straight line. In addition, a straight line connecting the end part 102A of the belt 102, the stopper 114, and the loop 106 intersects with a straight line connecting the end part 112A of the belt 112, the stopper 114, and the loop 116.

### Mounting of Coil Unit

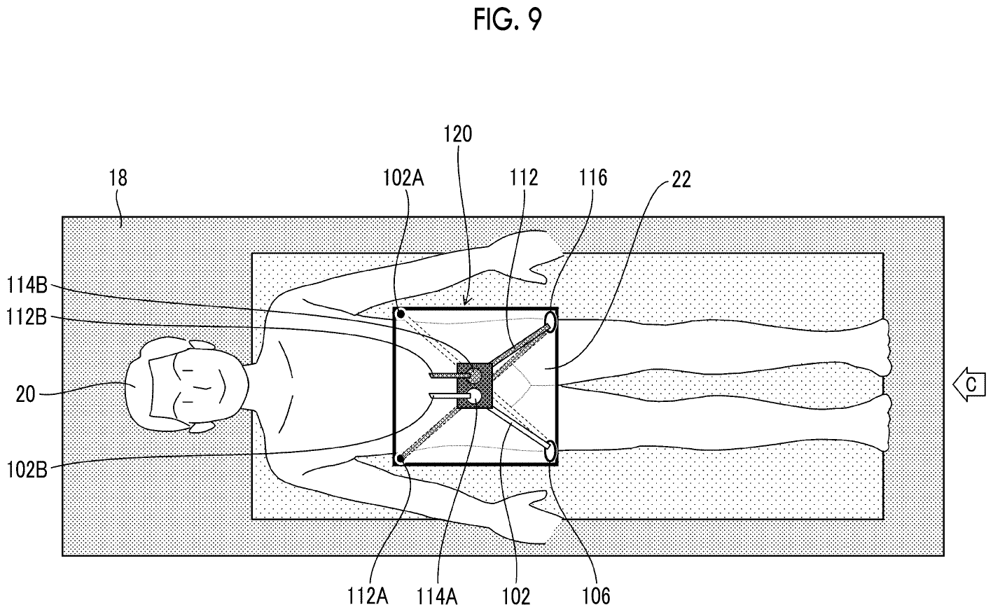

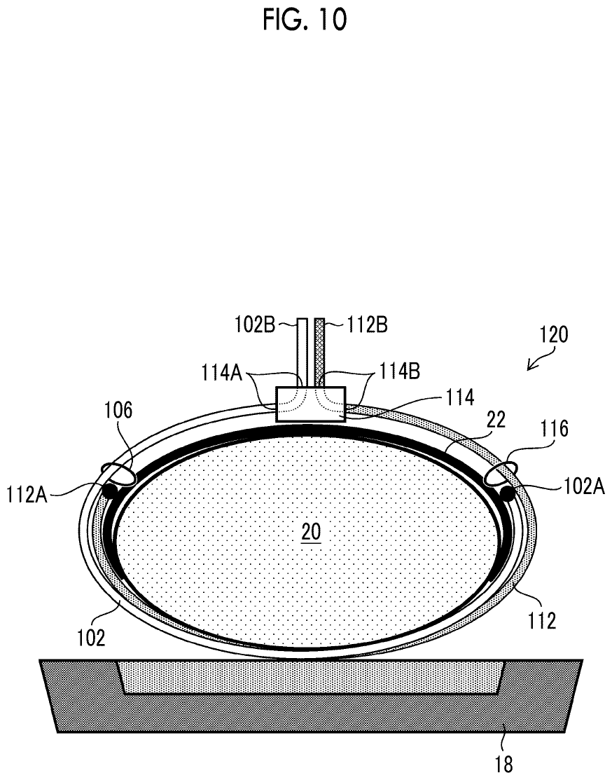

FIG. 9 is a top view of the patient 20 placed on the top plate 18. An abdominal coil unit 120 is mounted on an abdomen of a patient 20 shown in FIG. 9. In addition, in FIG. 9, the belt 102 and the belt 112 on the back side of the patient 20 are shown by a dotted line. In addition, FIG. 10 is a schematic view viewed from an arrow C of FIG. 9.

In order to mount the abdominal coil unit 120 on the patient 20 as shown in FIGS. 9 and 10, first, the user winds the belt 102 diagonally around the coil 22 through the lower surface side of the coil 22 and inserts the belt 102 from the end part 102B into the insertion hole 106A of the loop 106. In addition, the user inserts the belt 102 from the end part 102B into the insertion hole 114A of the stopper 114 that is unlocked state.

Similarly, the user winds the belt 112 diagonally around the coil 22 through the lower surface side of the coil 22 and inserts the belt 112 from the end part 112B into the insertion hole 116A of the loop 116. In addition, the user inserts the belt 112 from the end part 112B into the insertion hole 114B of the stopper 114 that is unlocked state. In this manner, the belt 102 and the belt 112 are wound around the lower surface side of the coil 22 in an opposite direction and intersect with each other.

Then, the user pulls the belt 102 and the belt 112 in the +radial direction to bring the coil 22 into a state of sufficiently attaching to the abdomen of the patient 20, and brings the locking mechanism of the insertion hole 114A and the locking mechanism of the insertion hole 114B of the stopper 114 to the locked state. Accordingly, the belt 102 and the belt 112 are fixed by the stopper 114, and the coil 22 is tightly fixed to the abdomen of the patient 20 by the belt 102 and the belt 112.

The user may bring the lower surface of the coil 22 into contact with the abdomen of the patient 20 and then wind the belt 102 and the belt 112 around the patient 20 to mount the abdominal coil unit 120 on the patient 20.

In this manner, with the abdominal coil unit 120 according to the third embodiment, the belt 102 and the belt 112 are fixed to both ends of the coil 22, and the belt 102 and the belt 112 are intersected and put on the patient 20. Accordingly, fixing of the coil 22 to the trunk of the patient 20 can be completed equally to the right and left by simply pulling the belt 102 and the belt 112 located on the upper surface of the coil 22 at positions that are easily gripped and held.

In the example shown in FIG. 9, although the abdominal coil unit 120 is mounted by aligning the coil 22 in the direction in which the ±axis direction in FIG. 8 is the trunk direction of the patient 20, the abdominal coil unit 120 may be mounted by aligning the coil 22 in the direction in which the ±circumferential direction in FIG. 8 is the trunk direction of the patient 20. In this manner, with the abdominal coil unit 120, the coil 22 can be brought into contact with the patient 20 more closely regardless of whether the coil 22 is used in the longitudinal direction or the lateral direction with respect to the trunk direction of the patient 20.

### Fourth Embodiment

### The Coil Fixed by the Coil Unit May be a Flexible Coil.

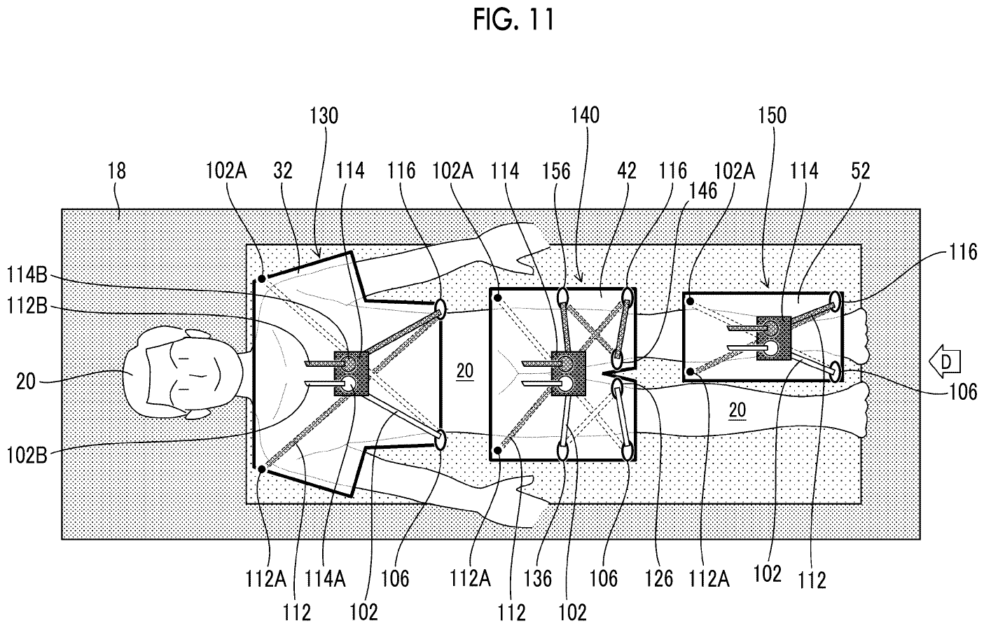

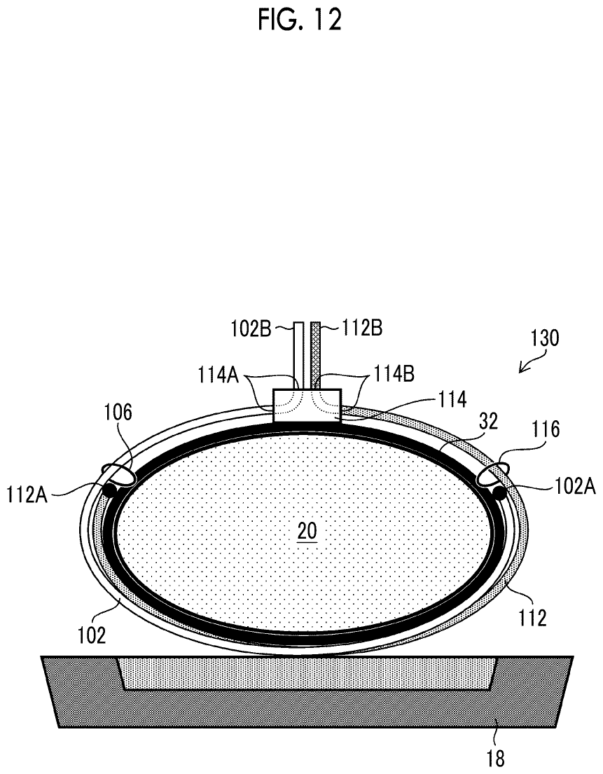

FIG. 11 is a top view of the patient 20 placed on the top plate 18. A chest coil unit 130 is mounted on a chest part of a patient 20 shown in FIG. 11, a waist coil unit 140 is mounted on a waist part, and a leg coil unit 150 is mounted on a left leg part. In addition, FIG. 12 is a schematic view viewed from an arrow D of FIG. 11, and shows the chest coil unit 130.

The chest coil unit 130 comprises a coil 32, a belt 102, a loop 106, a belt 112, a stopper 114, and a loop 116. The coil 32 is a wearable type and has a structure that encircles around the trunk of the patient 20 from the chest to the back as shown in FIG. 12.

As shown in FIG. 11, the belt 102, the loop 106, the belt 112, the stopper 114, and the loop 116 are disposed on the outer peripheral surface of the coil 32. The belt 102 and the belt 112 are intersected and put on the back side of the patient 20 in the same manner as the abdominal coil unit 120, and are fixed by the stopper 114. Accordingly, the chest coil unit 130 can bring the coil 32 into close contact with the patient 20.

The waist coil unit 140 comprises the coil 42, and comprises the belt 102, the loop 106, the belt 112, the stopper 114, the loop 116, the loop 126, the loop 136, and the loop 146, and the loop 156 on an outer peripheral surface of the coil 42. The coil 42 is a wearable type and has a structure that encircles around the trunk on the waist part of the patient 20.

As shown in FIG. 11, the belt 102 is wound diagonally of the coil 42 from the end part 102A to the back side of the patient 20 and passed through the loop 106. The belt 102 is wound around a front side of the right thigh part of the patient 20 and passed through the loop 126, and is wound around a back side of the right thigh part of the patient 20 and passed through the loop 136. Further, the belt 102 is wound around a front side of the right thigh part of the patient 20 and fixed by the stopper 114.

Similarly, the belt 112 is wound diagonally of the coil 42 from the end part 112A to the back side of the patient 20 and passed through the loop 116. The belt 112 is wound around a front side of the left thigh part of the patient 20 and passed through the loop 146, and is wound around a back side of the left thigh part of the patient 20 and passed through the loop 156. Further, the belt 112 is wound around a front side of the left thigh part of the patient 20 and fixed by the stopper 114. Accordingly, the waist coil unit 140 can bring the coil 42 into close contact with the patient 20.

The leg coil unit 150 comprises the coil 52, and comprises the belt 102, the loop 106, the belt 112, the stopper 114, and the loop 116 on an outer peripheral surface of the coil 52. The coil 52 is a wearable type and has a structure that encircles around the trunk on the leg part of the patient 20.

The belt 102 and the belt 112 are intersected and put on the back side of the patient 20, and are fixed by the stopper 114. Accordingly, the leg coil unit 150 can bring the coil 52 into close contact with the patient 20.

In this manner, with the chest coil unit 130, the waist coil unit 140, and the leg coil unit 150 according to the fourth embodiment, by fixing the belt to at least one point of each coil and putting the belt on the patient, the coils having a complicated shape and the wearable type coil can be brought into close contact with the patient.

### Fifth Embodiment

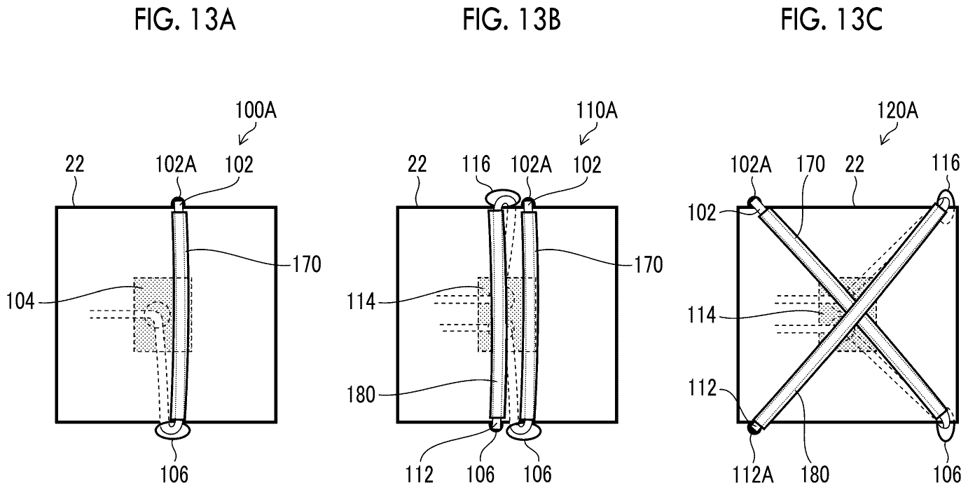

FIGS. 13A to 13C are diagrams respectively showing abdominal coil units 100A, 110A, and 120A according to a fifth embodiment. FIG. 13A shows an abdominal coil unit 100A. The abdominal coil unit 100A comprises a pipe 170 in addition to the abdominal coil unit 100.

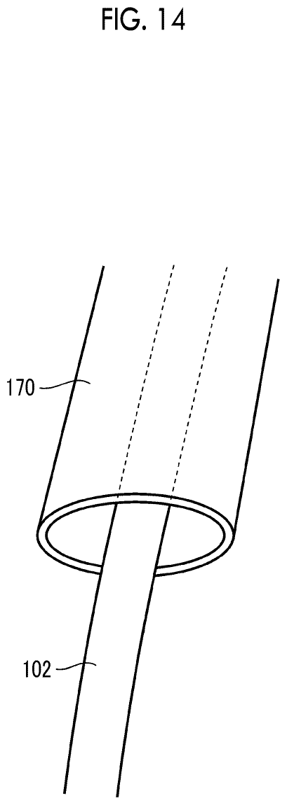

FIG. 14 is a partially enlarged view of the pipe 170. As shown in FIG. 14, a pipe 170 (an example of a “pipe member”) is a tubular member having an inner diameter larger than the belt 102. The pipe 170 may be a flexible material or a rigid material. The pipe 170 may have any of a true cylindrical shape, an elliptical cylindrical shape, or a corner cylindrical shape, but preferably has a flat shape as an outer shape.

As shown in FIG. 13A, the abdominal coil unit 100A passes the belt 102 of the abdominal coil unit 100 through the pipe 170 between the end part 102A of the belt 102 and the loop 106.

The pipe 170 is disposed on a back side of the patient in a case where the abdominal coil unit 100A is mounted on the patient. That is, the pipe 170 is disposed between the patient and the top plate 18 in a state in which the patient lies on the top plate 18. Therefore, even in a state in which the load of the patient is applied to the pipe 170, the belt 102 can be easily moved in the pipe 170. In addition, by making the outer shape of the pipe 170 flat, it is possible to alleviate the discomfort of the patient even at a position where the pipe 170 comes into contact with the patient.

FIG. 13B shows the abdominal coil unit 110A. The abdominal coil unit 110A comprises the pipe 170 and a pipe 180 in addition to the abdominal coil unit 110. The abdominal coil unit 110A passes the belt 102 of the abdominal coil unit 110 through the pipe 170 between the end part 102A of the belt 102 and the loop 106, and passes the belt 112 through the pipe 180 (an example of a “pipe member”) between the end part 112A of the belt 112 and the loop 116.

The pipe 170 and the pipe 180 are disposed on the back side of the patient in a case where the abdominal coil unit 110A is mounted on the patient. That is, the pipe 170 and the pipe 180 are disposed between the patient and the top plate 18 in a state in which the patient lies on the top plate 18. Therefore, even in a state in which the load of the patient is applied to the pipe 170 and the pipe 180, the belt 102 can be easily moved in the pipe 170, and the belt 112 can be easily moved in the pipe 180.

FIG. 13C shows an abdominal coil unit 120A. The abdominal coil unit 120A comprises the pipe 170 and the pipe 180 in addition to the abdominal coil unit 120. The abdominal coil unit 120A passes the belt 102 of the abdominal coil unit 120 through the pipe 170 between the end part 102A of the belt 102 and the loop 106, and passes the belt 112 through the pipe 180 between the end part 112A of the belt 112 and the loop 116.

The abdominal coil unit 120A also has the same effect as the abdominal coil unit 110A.

With the abdominal coil unit 110A, the abdominal coil unit 120A, and the abdominal coil unit 130A according to the fifth embodiment, by passing each belt at a portion passing through the back side of the patient through the flat pipe member, the belt can be easily moved even in a state in which the load of the patient lying on the examination table is applied, and the discomfort of the patient can be alleviated even in a case in which the belt comes into contact with the pipe.

### Sixth Embodiment

The belt 102 and the belt 112 according to the first embodiment to the fifth embodiment may be a reception side cable that outputs the magnetic resonance signal received by the coil. Accordingly, the number of components of the coil can be reduced, and the complexity of the structure can be suppressed.

Further, the reception side cables serving as the belt 102 and the belt 112 may be flexible flat cables in which a plurality of cables are bundled in a planar shape (belt shape).

As a result, it is possible to reduce the discomfort of the patient.

### Others

The technical scope of the present invention is not limited to the scope described in the embodiments. The configurations and the like in each embodiment can be appropriately combined between the respective embodiments without departing from the gist of the present invention.

### EXPLANATION OF REFERENCES

10: MRI apparatus
12: gantry
14: bore
16: examination table
18: top plate
20: patient
22: multi-channel RF receive coil (coil)

What is claimed is:

1. A coil unit comprising:
a receive coil that receives a magnetic resonance signal of an examination target;
a belt member with one end side fixed to the receive coil and the other end side being a free end; and
a locking member that is fixed to the receive coil and fixes the belt member at a position of any length from the one end side of the belt member.

2. The coil unit according to claim 1, further comprising:
a loop member that is fixed to the receive coil and restricts a path of the belt member.

3. The coil unit according to claim 2,
wherein the one end side of the belt member, the locking member, and the loop member are disposed in a straight line.

4. The coil unit according to claim 1,
wherein the belt member includes a first belt member of which one end side is fixed to the receive coil and the other end side is a free end, and a second belt member of which one end side is fixed to the receive coil and the other end side is a free end, and
the locking member includes a first locking member that fixes the first belt member and a second locking member that fixes the second belt member.

5. The coil unit according to claim 4, further comprising:
a first loop member that is fixed to the receive coil and restricts a path of the first belt member; and
a second loop member that is fixed to the receive coil and restricts a path of the second belt member.

6. The coil unit according to claim 5,
wherein a straight line connecting the one end side of the first belt member and the first loop member intersects with a straight line connecting the one end side of the second belt member and the second loop member.

7. The coil unit according to claim 1,
wherein the receive coil has a structure that encircles around a trunk of a patient.

8. The coil unit according to claim 1, further comprising:
a pipe member into which the belt member is inserted.

9. The coil unit according to claim 1,
wherein the locking member is fixed to a center of the receive coil.

10. The coil unit according to claim 1,
wherein the belt member is a cable that outputs the magnetic resonance signal received by the receive coil.

11. The coil unit according to claim 10,
wherein the belt member is a flat cable in which a plurality of cables are bundled in a planar shape.
